(19) United States
(12) Patent Application Publication
(10) Pub. No.: US 2024/0115287 A1
(43) Pub. Date: Apr. 11, 2024

(54) NECTROTIC TISSUE DEBRIDEMENT DEVICE

(71) Applicant: BOSTON SCIENTIFIC SCIMED, INC., Maple Grove, MN (US)

(72) Inventors: Enda Connaughton, Galway (IE); Charlene Deane, Roscommon (IE); Aiden Flanagan, Kilcolgan (IE); Richard Crawford, Galway (IE); Martin Lawrence Fawdry, Galway (IE); Michael Hughes, Galway (IE); Anthony O'Brien, Galway (IE)

(21) Appl. No.: 18/483,325

(22) Filed: Oct. 9, 2023

Related U.S. Application Data
(60) Provisional application No. 63/414,731, filed on Oct. 10, 2022.

(51) Int. Cl. A61B 17/32; A61B 90/00

(52) U.S. Cl. CPC A61B 17/32002; A61B 90/361; A61B 2017/00743; A61B 2017/320024

(57) ABSTRACT

The present disclosure provides a tissue debridement device and describes an example treatment for ANP using the tissue debridement device. The tissue debridement device has a sheath, a distal tip, and a plurality of rotating tines coupled to the distal tip and the sheath. The distal tip can be adjusted to be closer or further from the sheath to change a diameter of the rotating tines, thereby changing a cutting diameter of the tissue debridement device.

### CROSS-REFERENCE TO RELATED APPLICATIONS

This application claims the benefit of U.S. Provisional Patent Application Ser. No. 63/414,731 filed Oct. 10, 2022, the disclosure of which is incorporated herein by reference.

### TECHNICAL FIELD

The present disclosure generally relates to necrotic tissue debridement. Particularly, but not exclusively, the present disclosure relates to debridement of pancreatic pseudocyst necrotic tissue.

### BACKGROUND

In the United States the incidence of pancreatitis is approximately 185,000 cases per year. At least 80% of cases are due to alcohol and cholelithiasis. Acute necrotic pancreatitis (ANP) is reported by some to occur in approximately 20% of all episodes of pancreatitis. ANP is often treated with an endoscopic necrosectomy, which is performed by passing a flexible endoscope transorally and then transmurally into the necrotic cavity. After the puncture of the stomach or duodenal wall into the cystic cavity, the tract is dilated by a balloon and multiple large-bore double-pigtails stents or removable self-expanding stents such are placed. Debridement and lavage are carried out by a variety of devices (snares, baskets, balloons, forceps, nets and irrigation). Often, 3 to 6 sessions are necessary to achieve sufficient or complete debridement of the necrotic tissue.

The outcomes of endoscopic necrosectomy are encouraging. However, endoscopic necrosectomy suffers several limitations or disadvantages, such as, the need for multiple repeated procedures, which requires multiple sessions of sedation or anesthesia. Additionally, the necrotic tissue being debrided is difficult to grasp and remove from the cavity, which can lead to multiple, long, frustrating sessions before healthy granulation tissue can be seen. Further, the quantification of the necrotic burden, how to manage a large burden of necrotic tissue, how to manage deep retroperitoneal extension, and the difficulty, or impossibility, of treating the distal left-sided collections are other disadvantages. Moreover, endoscopic necrosectomy has technical limitations, such as, the lack of dedicated instruments, the difficulty to fix the bowel lumen to the cavity wall with staples or sutures, difficulty in avoiding vital structures (e.g., vessels, or the like) within the necrotic cavity.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

With some embodiments, the disclosure can be implemented as a tissue debridement device. The tissue debridement device can comprise a sheath; a distal tip; and a plurality of rotating tines coupled to the distal tip and the sheath.

In further embodiments, the tissue debridement device can comprise an aspiration port disposed on a distal end of the sheath.

In further embodiments of the tissue debridement device, the rotating tines configured to morcellate tissue.

In further embodiments of the tissue debridement device, the rotating tines configured to create, via rotation, a flow of the morcellated tissue towards the aspiration port.

In further embodiments, the tissue debridement device can comprise an infusion port.

In further embodiments of the tissue debridement device, the distal tip is atraumatic.

In further embodiments of the tissue debridement device, the distal tip comprises a probe configured to indicate healthy tissue.

In further embodiments of the tissue debridement device, each of the rotating tines comprise a sharp section disposed between blunt sections.

In further embodiments of the tissue debridement device, the rotating tines comprise a wire and wherein the sharp section comprises a smaller diameter than the blunt sections.

In further embodiments of the tissue debridement device, the rotating tines comprise a wire and wherein the blunt sections comprise a coating.

In further embodiments of the tissue debridement device, the rotating tines comprise a wire and wherein the sharp section has a flat or tear shaped profile.

In further embodiments of the tissue debridement device, the distance between the distal tip and the sheath can be adjusted to adjust a diameter of the rotating tines.

In further embodiments, the tissue debridement device can comprise a central guide coupling the distal tip with the sheath, the central guide coupled to a handle at a proximal end of the sheath, the distance between the distal tip and the sheath adjustable by the handle.

In further embodiments of the tissue debridement device, the sheath is configured to be inserted into a working channel of an endoscope.

In further embodiments of the tissue debridement device, the distal tip comprises a camera.

With some embodiments, the disclosure can be implemented as a method for debriding tissue. The method can comprise introducing a tissue debridement device into a cystic cavity, the tissue debridement device comprising a sheath, a distal tip, and a plurality of rotating tines coupled to the distal tip and the sheath; adjusting a cutting diameter of the rotating tines of the tissue debridement device; and activating a motor drive unit coupled to the tissue debridement device to morcellate necrotic tissue in the cystic cavity.

In further embodiments of the method, the rotating tines comprise a wire and the sharp section comprises a smaller diameter than the blunt sections.

In further embodiments of the method, the rotating tines comprise a wire and the blunt sections comprise a coating.

In further embodiments of the method, the rotating tines comprise a wire and the sharp section has a flat or tear shaped profile.

With some embodiments, the disclosure can be implemented as a system. The system can comprise a tissue debridement device, the tissue debridement device comprising a sheath; a distal tip; and a plurality of rotating tines coupled to the distal tip and the sheath; and an endoscope configured to receive the tissue debridement device.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a tissue debridement device.

FIG. 2A and FIG. 2B illustrates the tissue debridement device of claim 1 in alternative detail.

FIG. 3 illustrates a tissue debridement device.

FIG. 4 illustrates a tissue debridement device.

FIG. 5 illustrates a tissue debridement device.

FIG. 6 illustrates a tissue debridement device.

FIG. 7 illustrates a tissue debridement device.

FIG. 8 illustrates a method.

FIG. 9 illustrates a procedure environment.

### DETAILED DESCRIPTION

As introduced above, the present disclosure provides a tissue debridement device and described an example treatment for ANP using the tissue debridement device. The tissue debridement device has a sheath, a distal tip, and a plurality of rotating tines coupled to the distal tip and the sheath. The distal tip can be adjusted to be closer or further from the sheath to change a diameter of the rotating tines, thereby changing a cutting diameter of the tissue debridement device.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

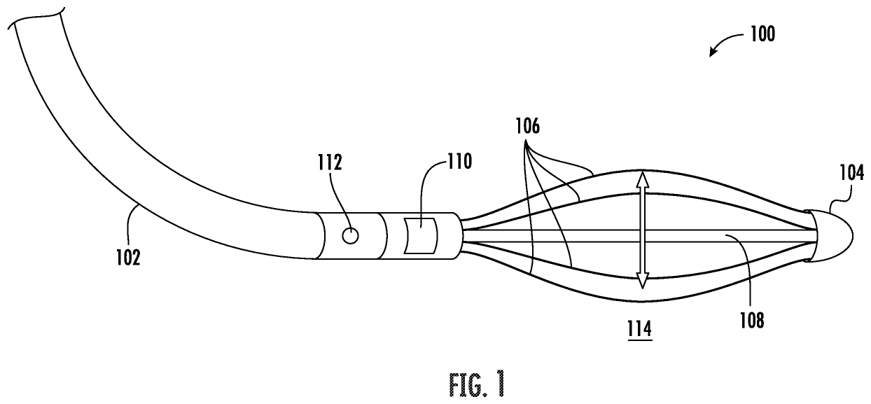

FIG. 1 illustrates a tissue debridement device 100, according to some embodiments of the present disclosure. The tissue debridement device 100 includes a sheath 102 and a distal tip 104 coupled to the sheath 102 via rotating tines 106 and a central guide 108. The sheath 102 can be any of a variety of materials, such as, for example, an elastomer material such as, polyurethane elastomer, polyester elastomer, or the like.

The rotating tines 106 can be arranged to rotate axially to debride necrotic tissue as outlined herein. Said differently, the rotating tines 106 can be arranged to rotate about a longitudinal axis of the tissue debridement device 100 such that the rotating tines 106 cuts, morcellates, and/or otherwise debrides necrotic tissue. The central guide 108 can be used to change a cutting diameter 114 of the rotating tines 106. For example, the central guide may be coupled to a handle disposed on a proximal end of the sheath 102. The handle can be configured to move the distal tip 104 closer or further from the sheath 102 to change the cutting diameter 114. Further, the central guide 108 can be configured to stabilize the distal tip 104 and the rotating tines 106 during use. For example, the rotating tines 106 can be formed of thinner material, relative to the central guide 108, such that the tines are sharper or able to cut through tissue. With some examples, the rotating tines 106 can be formed from a single wire, from braided wire, or from flattened wire. In some embodiments, the rotating tines 106 can be polymer or plastic material and may be oval shaped, tear drop shaped, or flattened. Where the rotating tines 106 are tear shaped, the narrow pointed portion of the tear shape can be disposed in the direction of rotation. This is described in greater detail below.

Although not shown herein, the rotating tines 106 can be coupled (e.g., via a driveshaft, or the like) to a motor drive unit at a proximal end of the sheath 102. The motor drive unit can drive the axial rotation of the rotating tines 106 during use. In such a manner, the rotation of the blades or tines be configured to feed the morcellated necrotic tissue towards an aspiration port. For example, tissue debridement device 100 can include aspiration port 110 in which morcellated tissue is removed via suction. With some examples, aspiration port 110 can be coupled to a vacuum pump disposed at a proximal end of sheath 102. The vacuum pump can create a suction to assist in drawing the morcellated tissue from the area of debridement into the stomach, into a clinical waste trap, or the like.

Tissue debridement device 100 can further include an infusion port 112. Infusion port 112 can be coupled to a fluid reservoir and pump system and be configured to infuse or irrigate the area of debridement with a fluid, such as, for example water, saline, hydrogen peroxide, or the like. With some examples, the fluid can be heated, be cooled, or be left at room temperature. In some examples, infusion port 112 can be located at the distal tip of 104 of the tissue debridement device so that a pressure gradient can be created from the distal tip 104 to the aspiration port 110, thereby creating a flow of morcellated necrotic tissue to be aspirated towards port 110.

With some examples, the distal tip 104 can be configured to be atraumatic (e.g., can be blunt, rounded, smooth, or the like). In some embodiments, the distal tip 104 can include a sensor or probe configured to indicate healthy tissue. For example, the distal tip 104 can be configured with a depth guide arranged to provide feedback (e.g., to a physician, or the like) when the border between healthy and necrotic tissue is encountered by the distal tip 104.

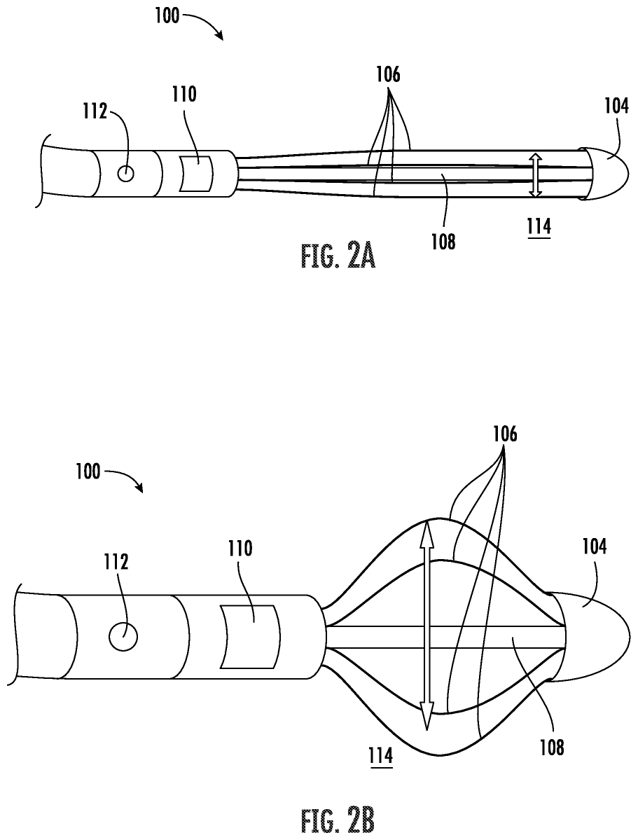

FIG. 2A and FIG. 2B illustrates the tissue debridement device 100 where the cutting diameter 114 is adjusted to different diameters. FIG. 2A illustrates the tissue debridement device 100 in a collapsed or extended state in which the central guide 108 is moved further from the proximal end of the sheath 102 such that the distal tip 104 is moved distally along the longitudinal axis of the sheath 102 and away from the distal end of the sheath 102 to stretch out the rotating tines 106 and reduce the cutting diameter 114.

Conversely, FIG. 2B illustrates the tissue debridement device 100 in an expanded or shortened state in which the central guide 108 is moved closer to the proximal end of the sheath 102 such that the distal tip 104 is moved proximally along the longitudinal axis of the sheath 102 and closer to the distal end of the sheath 102 to expand the rotating tines 106 and increase the cutting diameter 114.

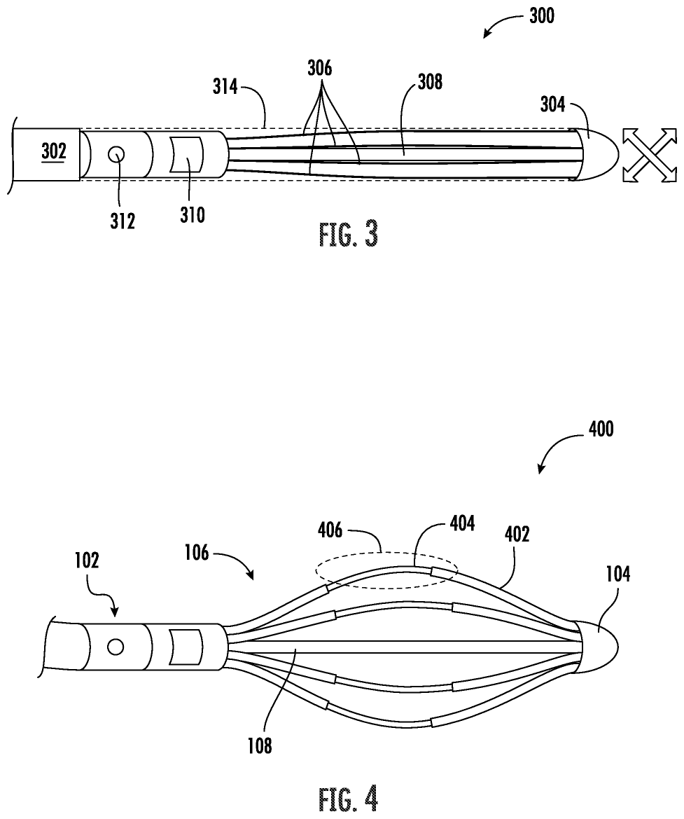

With some implementations, the sheath 102 can be configured (e.g., sized and/or shaped) to be inserted into a working channel of an endoscope. For example, the tissue debridement device 100 depicted in FIG. 1 can be configured for insertion into an endoscope where steering and vision are provided. In other examples, the device can be independently steerable and/or include a camera, light, or other visualization features. FIG. 3 illustrates a tissue debridement device 300, according to some embodiments of the present disclosure. Tissue debridement device 300 includes a sheath 302 and a distal tip 304 coupled to the sheath 302 via rotating tines 306 and a central guide 308. The tissue debridement device 300 further includes aspiration port 310 and infusion port 312. Tissue debridement device 300 can be arranged to steer independent, or rather, rotating tines 306 can move in directions relative to the longitudinal axis of the sheath 302. For example, central guide 308 may include steering mechanisms and structure like a conventional endoscope. Further, with some examples, distal tip 304 can include a camera and/or light to provide vision. With some examples, the camera can provide a view distal to the distal tip 304. In some examples, the camera can provide a view proximal to the distal tip 304, or rather, back towards the sheath 302. With further embodiments, the camera can provide a view angled away from the longitudinal axis of the sheath 302, such that the camera provides a view of the region in which the rotating tines 306 will contact tissue. In some examples, the camera can provide both a distal and proximal view from the distal tip 304.

In some embodiments, retractable sheath 314 can be retracted back into sheath 302 to expose rotating tines 306. For example, retractable sheath 314 can be extended out from sheath 302 towards distal tip 304 to cover rotating tines 306 and the tissue debridement device 300 can be inserted transorally into a patient's stomach and then transmurally into a necrotic cavity (e.g., a portion of the biliary system, or the like). As such, the retractable sheath 314 can prevent accidental cutting or damage to the patient during insertion. Once the distal end of tissue debridement device 300 is inserted into the necrotic cavity, the retractable sheath 314 can be retracted exposing the rotating tines 306 and the rotating tines 306 can be adjusted such that a desired cutting diameter is realized.

With some implementations, the entire rotating tines are “sharp” cutting features. In other examples, only a portion of the rotating tines can be configured to debride tissue. For example, FIG. 4 illustrates a tissue debridement device 400, according to some embodiments of the present disclosure. Tissue debridement device 400 includes rotating tines 106 like the tissue debridement device 100 of FIG. 1. However, the rotating tines 106 includes a blunted section 402 and a sharp section 404 which when rotated proves a cutting region 406. As noted above the rotating tines 106 can be formed from a wire, a braided wire, a flattened wire, a plastic components, or the like. In some examples, the rotating tines 106 can be a partially coated wire. In particular, the blunted section 402 can be coated (e.g., with plastic, a polymer, or the like) while the sharp section 404 can be exposed or uncoated. In other implementations, the blunted section 402 can be a larger diameter relative to the sharp section 404. With still other implementations, the blunted section 402 can be un-flattened while the sharp section 404 can be flattened. In still another example, the blunted section 402 can be circular or oval shaped while the sharp section 404 can be tear shaped. For example, where the sharp section 404 is tear shaped, the cross section of the tine can be tear shaped (or an aero foil shape) where the narrow section of the cross section of the tear shape faces the direction of rotation.

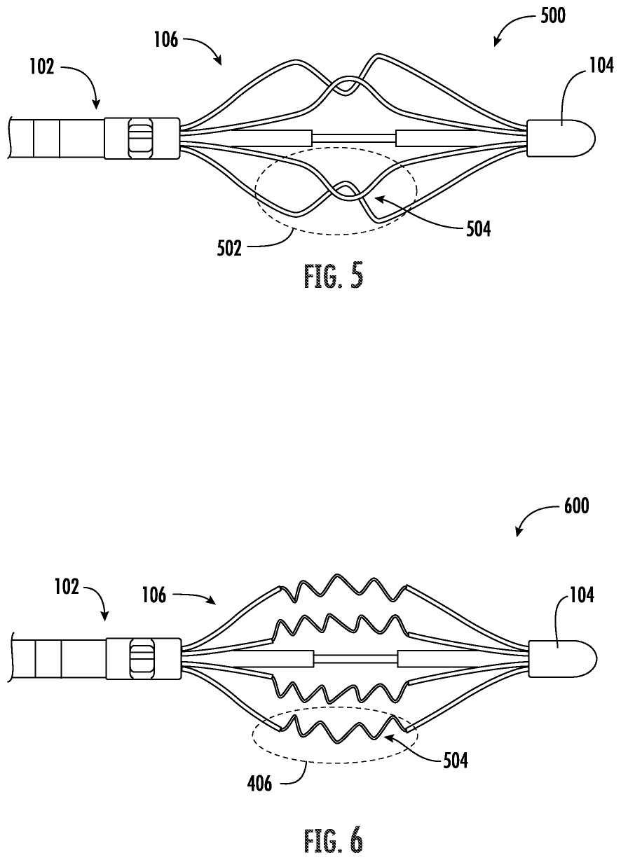

With some implementations, the rotating tines can be arranged to “tear” or pull the tissue as opposed to “cut” the tissue. For example, FIG. 5 illustrates a tissue debridement device 500, according to some embodiments of the present disclosure. Tissue debridement device 500 includes rotating tines 106 like the tissue debridement device 100 of FIG. 1. However, the rotating tines 106 includes a tearing region 502 where the rotating tines 106 are configured with a v shape 504. With some embodiments, the rotating tines 106 comprise the v shape 504 (or otherwise concave shape) arranged to pinch, pull, tease, or tear away the necrotic tissue from the healthy tissue as opposed to cutting the necrotic tissue away.

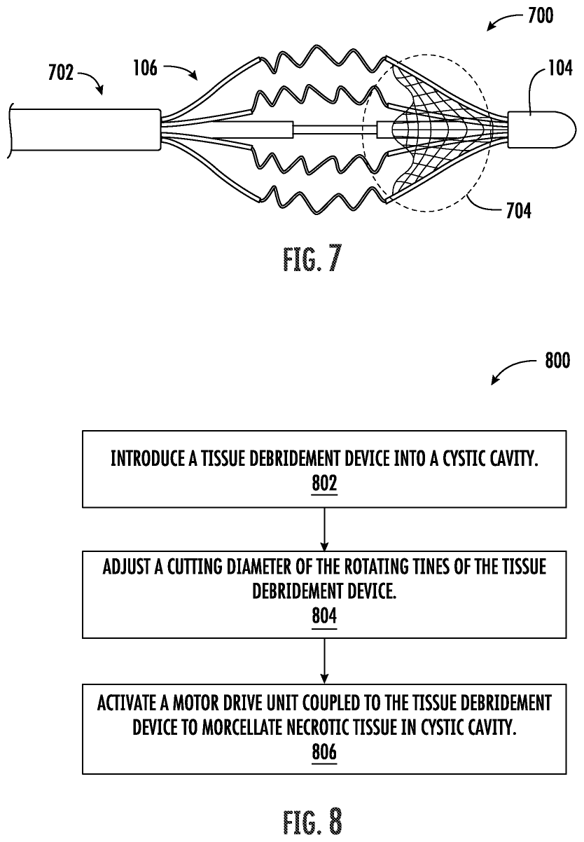

With some embodiments, the rotating tines 106 includes multiple concave or v shaped grooves. For example, FIG. 6 illustrates a tissue debridement device 600, according to some embodiments of the present disclosure. Tissue debridement device 600 includes rotating tines 106 like the tissue debridement device 500 of FIG. 5. However, the rotating tines 106 includes multiple v shaped grooves 504 in the tearing region 502.

In some embodiments, the tissue debridement devices described herein can be configured without an aspiration port. For example, FIG. 7 illustrates a tissue debridement device 700, according to some embodiments of the present disclosure. Tissue debridement device 700 includes a sheath 702 without an aspiration port. In such an embodiment, necrotic tissue can be gathered and pulled out of the necrotic cavity into the stomach using an umbrella 704. The umbrella 704 can be formed from a mesh, net, or membrane and configured to gather necrotic tissue that is cut or removed from the cavity.

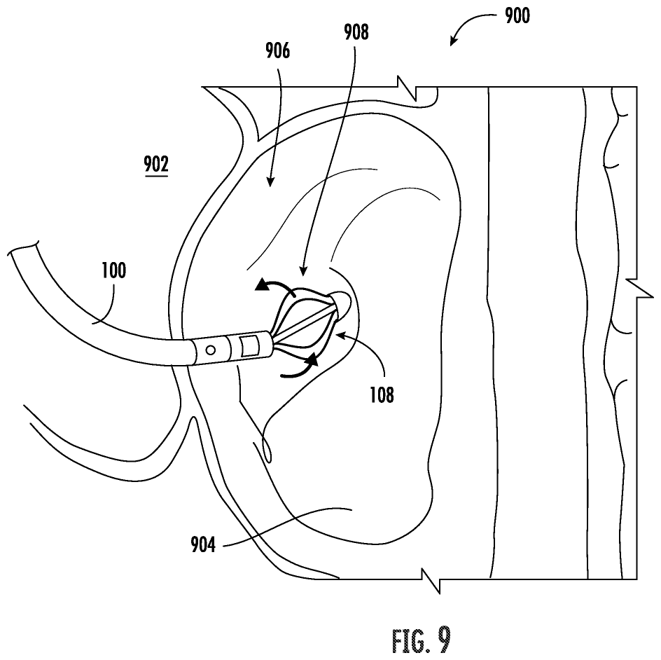

FIG. 8 illustrates a method 800 for removing necrotic tissue from a duct in the biliary tree of a patient. For example, method 800 can be implemented to treat acute necrotic pancreatitis. As such, the method 800 is described with reference to the patient biliary system 900 of FIG. 9, which shows stomach 902 and pancreas 904. The patient biliary system 900 further depicts a pancreatic pseudocyst 906 and necrotic tissue 908. It is to be appreciated, that pancreatic pseudocyst 906, also referred to as a necrotic cavity, can develop adjacent to the pancreas 904 because of alcohol or cholelithiasis as outlined above. As a result of the pancreatic pseudocyst 906, necrotic tissue 908 may develop.

Method 800 can begin at block 802. At block 802 a tissue debridement device is introduced into a cystic cavity. For example, tissue debridement device 100 can be introduced into pancreatic pseudocyst 906 at block 802. Often, the pancreatic pseudocyst 906 will have necrotic tissue 908. Further, the tissue debridement device (e.g., tissue debridement device 100, or the like) will have a sheath, a distal tip, and a plurality of rotating tines coupled to the distal tip and the sheath. Further, the tissue debridement device can have an aspiration port and/or an infusion port. With some examples, the tissue debridement device can be introduced into the necrotic cavity via an endoscope. In a specific example, the tissue wall between the stomach 902 and the pancreatic pseudocyst 906 can be fixed with a lumen opposing metal stent, such as, an Axios® stent.

Continuing to block 804 a cutting diameter of the tissue debridement device can be adjusted. For example, cutting diameter 114 of the tissue debridement device can be adjusted (e.g., via a handle and the central guide 108, or the like). Continuing to block 806 a motor drive unit can be activated to morcellate necrotic tissue in the cystic cavity. For example, a motor drive unit coupled to the rotating tines 106 can be activated to cause the rotating tines 106 to rotate to morcellate necrotic tissue 908. Rotation speed can be adjusted (e.g., at block 806) so that care is taken to avoid trauma to healthy tissue. In some embodiments, rotation speed can be less than or less than or equal to 2 rotations per minute (rpm), such as, for example, where delicate tissue structures (e.g., blood vessels, etc.) are proximate to the rotating tines 106.

In some examples, the tissue debridement device can include an aspiration port and/or an infusion port. As such, during the procedure, the pancreatic pseudocyst 906 can be irrigated and aspirated to remove, or aid removal of the morcellated necrotic tissue 908.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A tissue debridement device, comprising:
a sheath;
a distal tip; and
a plurality of rotating tines coupled to the distal tip and the sheath.

2. The tissue debridement device of claim 1, comprising an aspiration port disposed on a distal end of the sheath.

3. The tissue debridement device of claim 1, the rotating tines configured to morcellate tissue.

4. The tissue debridement device of claim 3, the rotating tines configured to create, via rotation, a flow of the morcellated tissue towards the aspiration port.

5. The tissue debridement device of claim 1, comprising an infusion port.

6. The tissue debridement device of claim 1, wherein the distal tip is atraumatic.

7. The tissue debridement device of claim 1, wherein the distal tip comprises a probe configured to indicate healthy tissue.

8. The tissue debridement device of claim 1, wherein each of the rotating tines comprise a sharp section disposed between blunt sections.

9. The tissue debridement device of claim 8, wherein the rotating tines comprise a wire and wherein the sharp section comprises a smaller diameter than the blunt sections.

10. The tissue debridement device of claim 8, wherein the rotating tines comprise a wire and wherein the blunt sections comprise a coating.

11. The tissue debridement device of claim 8, wherein the rotating tines comprise a wire and wherein the sharp section has a flat or tear shaped profile.

12. The tissue debridement device of claim 1, wherein the distance between the distal tip and the sheath can be adjusted to adjust a diameter of the rotating tines.

13. The tissue debridement device of claim 12, comprising a central guide coupling the distal tip with the sheath, the central guide coupled to a handle at a proximal end of the sheath, the distance between the distal tip and the sheath adjustable by the handle.

14. The tissue debridement device of claim 12, wherein the sheath is configured to be inserted into a working channel of an endoscope.

15. The tissue debridement device of claim 12, wherein the distal tip comprises a camera.

16. A method comprising:
introducing a tissue debridement device into a cystic cavity, the tissue debridement device comprising:
a sheath,
a distal tip, and
a plurality of rotating tines coupled to the distal tip and the sheath;
adjusting a cutting diameter of the rotating tines of the tissue debridement device; and
activating a motor drive unit coupled to the tissue debridement device to morcellate necrotic tissue in the cystic cavity.

17. The method of claim 16, wherein the rotating tines comprise a wire and wherein the sharp section comprises a smaller diameter than the blunt sections.

18. The method of claim 16, wherein the rotating tines comprise a wire and wherein the blunt sections comprise a coating.

19. The method of claim 16, wherein the rotating tines comprise a wire and wherein the sharp section has a flat or tear shaped profile.

20. A system comprising:
a tissue debridement device, the tissue debridement device comprising:
a sheath;
a distal tip; and
a plurality of rotating tines coupled to the distal tip and the sheath; and
an endoscope configured to receive the tissue debridement device.
